(19) United States
(12) Plant Patent Application Publication
(10) Pub. No.: US 2019/0223340 P1
(43) Pub. Date: Jul. 18, 2019

(54) Raspberry plant - 'ATENA' CULTIVAR

(50) ;

(71) Applicant: SANT'ORSOLA SOCIETA' COOPERATIVE AGRICOLA, Pergine Valsugana (IT)

(72) Inventors: Sara Giacomelli, Vigolo Vattaro (IT); Marco Giacomelli, Vigolo Vattaro (IT); Alessandra Viliotti, Vigolo Vattaro (IT)

(21) Appl. No.: 16/350,827

(22) Filed: Jan. 18, 2019

(30) Foreign Application Priority Data
Jan. 18, 2018 (QZ) 20180034

(51) Int. Cl. A01H 6/74

(52) U.S. Cl. PLT/204

(57) ABSTRACT

A new a primocane raspberry cultivar named ‘Atena’ having good fruit colour and flavour, large and very firm berries is disclosed. The new cultivar has the advantages of producing fruits that are very easy to pick and produces two crops per year, wherein the fruits mainly ripen on current season's cane in autumn.

Genus and species: Rubus idaeus L.

Variety denomination: ‘Atena’.

### BACKGROUND OF THE NEW VARIETY

A new and distinct cultivar of red raspberry (Rubus idaeus L.) hereinafter referred to by the cultivar denomination ‘Atena’ is disclosed. The new variety ‘Atena’ was obtained from the controlled cross between the varieties Dolomia Plus (mother plant) and Erika (father plant).

The crossing for the new cultivar occurred in year 2007 in Altopiano della Vigolana (Trento—Italy) and it has been subject to selection in 2008, and successively tested until 2017. The characteristics of the new cultivar have been found stable and have been transmitted without change through succeeding (more than 9 years) asexual propagations by root cutting.

Plant breeder's right were applied for with the European Union on Jan. 18, 2018: ‘Atena’ has not been made publicly available.

### SUMMARY OF THE INVENTION

The most outstanding and distinguishing characteristics of the ‘Atena’ cultivar are hereunder reported.
Primocane raspberry plant;
Good fruit colour and flavour;
Very firm light red fruits;
Easy to pick up (low adherence to plug).

The new variety produces two crops per year and the fruits mainly ripen on current season's cane in autumn.

The new variety can be propagated by: the use of suckers produced in a spawn-bed, root cutting and use of tissue culture techniques. The root cutting is to be preferred.

### BRIEF DESCRIPTION OF THE PHOTOGRAPHS

The new red raspberry cultivar ‘Atena’ is illustrated by the accompanying photographs showing typical specimens of the new variety in colour as true as reasonably possible to make the same colour illustrations of this character by conventional photographic procedures.

FIG. 1 is a view showing the raspberry plants with ripening fruit. The upright, tall plants and vigorous laterals can be appreciated.

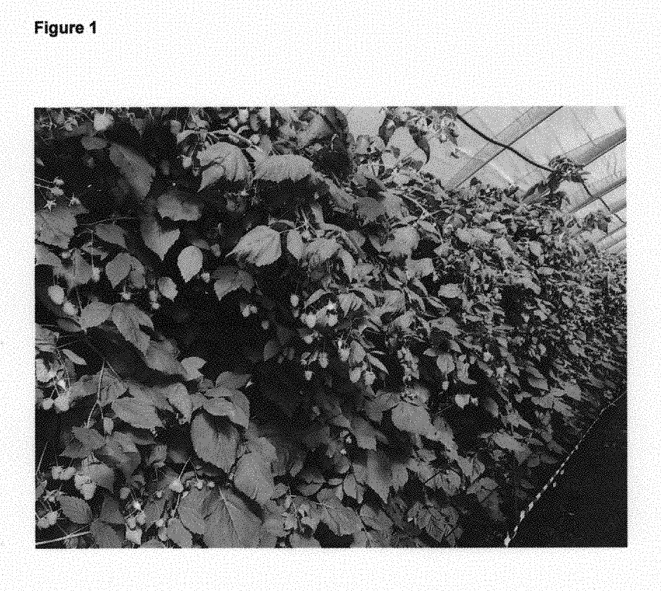

FIG. 2 is a view showing the leaf upper and lower surfaces.

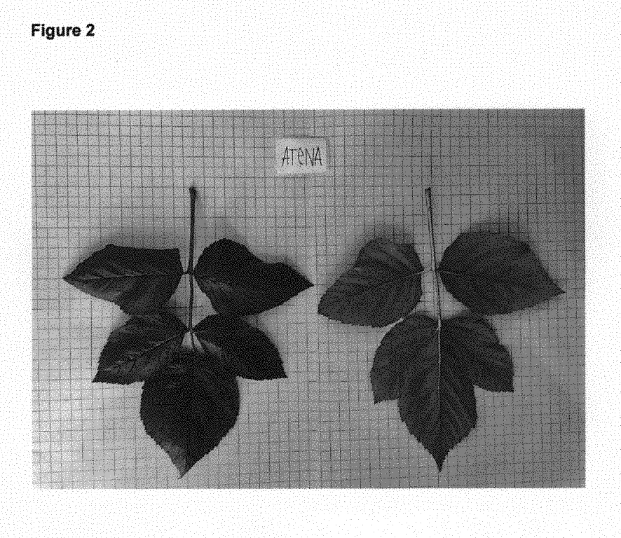

FIG. 3 is a view showing the plant primocane internode length and cane colour.

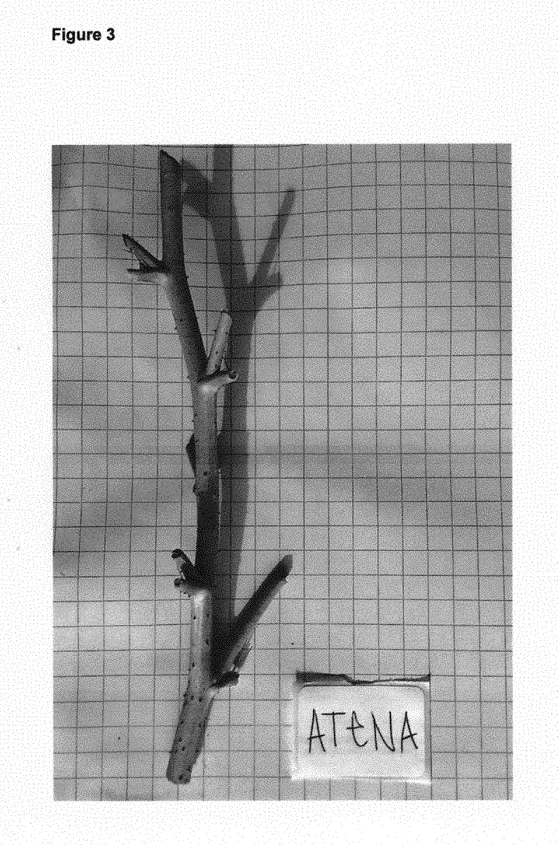

FIG. 4 is a view showing the ripe fruit size and shape.

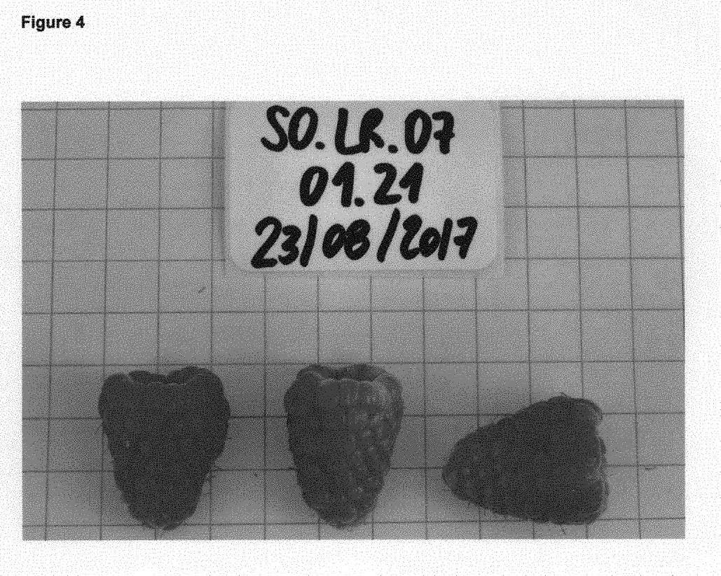

### DETAILED DESCRIPTION

The following detailed description set forth the distinctive characteristics of the new variety ‘Atena’. The performance of new cultivar for retention of its distinctive characteristics has been evaluated through successive asexual propagations by root cutting in the Applicant's setting in Trentino (Italy) under natural conditions in high tunnels. The growing was in soils using coir as substrate.

The plant assessment was performed for 2 years.
Classification:
   Family.—Rosaceae.
   Botanical.—Rubus idaeus L.
   Common name.—Raspberry.
Parentage: Dolomia Plus x Erika.
Plant:
   General habit.—Upright canes; strong vigor; good roots system; adapted to medium winter chill conditions such as those of Northern Italy; high productivity in autumn.
   Average height (average).—1.80-2 m.
   Spread (average).—0.90 m.
   Shape.—Conical.
   Vegetative bud length.—Medium 3.0-5.0 mm.
Primocane:
   Length (average).—1.80-2.0 m.
   Diameter (average) at 1.0 m height.—0.8-1.0 cm.
   Texture.—Smooth.
   Internode length (average) at 1.0 m height.—6-7 cm.
   Number of fruiting laterals per cane (average).—13-14.
   Number of current season's shoots.—Few to medium.
   Young shoot colour.—*RHS:140A. * Colour references made to R.H.S. Colour Chart of The Royal Horticultural Society of London (Edition V).
   Dormant cane colour.—*RHS:185A.
   Cane colour.—*RHS: N144C.
   Spines.—Present.
Spines:
   Shape.—Conical.
   Size (average).—1.0-1.5 mm.
   Density (average) at 1.2 m.—3 per cm².
   Colour.—*RHS 59A.
Foliage:
Leaves:
   Shape.—Compound; odd-pinnate and subdivided in 3-5 ovate and doubly serrate leaflets.
   Profile in cross section.—Concave.
   Rugosity.—Moderate.
   Complete leaf.—Length: 30.5 cm. Width: 19.8 cm. Number of leaflets: 3-5.
   Upper surface colour.—*RHS 139B.
   Lower surface colour.—*RHS 137D.
   Lateral leaflets.—Speculate.
   Lateral leaflets average size.—Length: 11.0 cm. Width: 6.5 cm.
   Lateral leaflets apex shape.—Pointed.
   Lateral leaflets base shape.—Ovate.
   Lateral leaflets margin type.—Doubly serrate.
   Terminal leaflets average size.—Size: Length: 13.5 cm. Width: 8.3 cm.
   Terminal leaflets apex shape.—Pointed.
   Terminal leaflets base shape.—Ovate.
   Terminal leaflets margin type.—Doubly serrate.
Petiole:
   Average length.—8.7 cm.
Flowers:
   Number per plant (average).—250.
   Length (average).—9.50 mm.
   Diameter (average).—30.35 mm.
   Diameter (receptacle).—8.10 mm.
   Shape.—Pentagonal.
   Petals colour.—*RHS NN155B.
   Fragrance.—Absent.
   Blooming.—July-August.
   Pedicel length (average).—2.5 cm.
Fruit:
   Predominant shape.—Elongated conical.
   Weight.—5.5 g.
   Size.—Length 2.7 cm. width 2.2 cm.
   Colour of ripe fruit.—*RHS 45D.
   Colour of immature fruit.—*RHS 41D.
   Firmness.—Very firm (0.06 kg).
   Drupes per fruit (average).—105.
   Drupe size.—Length: 3.9 mm. Width: 3.6 mm. Depth: 5.9 mm.
   Glossiness.—Medium.
   Adherence to plug.—Low.
   Titratable acidity.—10.22 meq/hg.
   Soluble solids.—7.6 ° Brix.
   Yield.—4.7 Kg/m (3 plants/m).
   Main bearing type.—On current year's cane in autumn from half August to late October.
   Storage capacity (shelf life).—Good shelf-life.
Reproductive organs:
   Pistil number per flower (average).—117.
   Pistil size (average).—4.41 mm.
   Stamen number per flower (average).—90.
   Stamen size (average).—4.82 mm.
Seeds:
   Size (average).—Length: 3.41 mm. width: 2.06 mm.
   Shape.—Kidney shaped.

### COMPARISON WITH PARENTAL AND COMMERCIAL CULTIVAR

Tale 1. Comparison table between variety and parents/other

| Characteristic | Evita | Atena |
|---|---|---|
| fruit color | Orange Red | Light red |
| Plant (size/vigour) | 1.70-1.90 m | 1.80-2.00 m |
| Fruit: firmness | Firm | Very firm |
| Fruit shape | Slight irregular | Very regular |
| Fruit picking | Slight difficult | Very easy |

What is claimed:

1. A new and distinct cultivar of raspberry plant as substantially herein shown and described.
